(19) United States
(12) Patent Application Publication
(10) Pub. No.: US 2012/0040906 A1
(43) Pub. Date: Feb. 16, 2012

(54) PROTEIN TARGETS IN DISEASE

(75) Inventors: Afshin Samali, Co. Galway (IE); Sanjeev Gupta, Co. Galway (IE)

(73) Assignee: NATIONAL UNIVERSITY OF IRELAND, GALWAY, Galway (IE)

(21) Appl. No.: 13/202,482

(22) PCT Filed: Feb. 26, 2010
(86) PCT No.: PCT/EP2010/052504

§ 371 Date: Oct. 31, 2011

(30) Foreign Application Priority Data
Feb. 26, 2009 (IE) 2009/0047

(51) Int. Cl. A61K 38/02; C40B 30/04; A61K 31/7105; G06F 19/18; C12Q 1/68; G01N 33/68; A61P 9/00; A61P 9/04; C07H 21/02; C07K 14/47

(52) U.S. Cl. 514/16.4; 536/23.1; 506/9; 514/44 R; 530/350; 514/1.1; 435/6.12; 436/86; 702/19

(57) ABSTRACT

MicroRNAs have been shown to be critically involved in control of cell survival and cell death decisions. By identifying microRNAs implicated in Endoplasmic Reticulum stress-induced cardiomyocyte apoptosis, it is envisaged that protein targets involved in same can be identified through specifically selected microRNAs. The microRNAs targeted are miR-351, miR-322, miR-125, miR-424 and miR-7a. Furthermore, the potential application of these identified proteins in the treatment of cardiovascular disease, in particular congestive heart failure, is disclosed.

### FIELD OF THE INVENTION

The present invention relates to a method of identifying protein targets implicated in Endoplasmic Reticulum stress-induced cardiomyocyte apoptosis and the application of these identified proteins in the treatment of cardiac disease, in particular congestive heart failure.

### BACKGROUND TO THE INVENTION

Heart disease is a leading cause of morbidity and mortality in the developed world. Cardiovascular disease (CVD), a group of disorders of the heart and the vasculature, includes high blood pressure, coronary heart disease, congestive heart failure, stroke and congenital heart defects. Heart failure is caused by any condition which reduces the efficiency of the myocardium, or heart muscle, through damage or overloading. The heart gets oxygen and nutrients through blood vessels called the coronary arteries. When the blood flow to the heart is cut off, the decrease in the supply of oxygen and nutrients causes lasting damage to myocardium. It is well documented that CVD leading to heart failure involves not only contractile dysfunction, but also cardiomyocyte death. Cell death is the end result of the convergence of multiple signalling pathways during CVD, triggered by events such as nutrient and oxygen deprivation, ion imbalance and excessive reactive oxygen species (ROS) production. Apoptosis has important pathophysiological consequences during Congestive Heart Failure (CHF), contributing to the loss of cardiomyocytes and functional abnormalities of the myocardium.

For example, Over 2 million people in the U.S. alone suffer from congestive heart failure (CHF) with over 400,000 new cases diagnosed every year. The most common cause of CHF is ischemic heart disease, which is the result of an acute or chronic lack of blood supply to the heart. In the ischemic state the lack of oxygen and nutrients to the heart can cause lasting damage to this vital organ through cardiomyocyte death.

Current approaches to the treatment of heart failure comprise maintaining an ideal body weight. Maintaining a healthy body weight can provide a 35-55% decrease in the risk of coronary heart disease. In this regard, obesity is perhaps second only to smoking as the leading avoidable cause of premature deaths. Further, maintenance of an active lifestyle is associated with a 35-55% lower risk of coronary heart disease.

Many different medications are used in the treatment of heart failure. They include:

Angiotensin-converting enzyme inhibitors (ACEI): Angiotensin-converting enzyme (ACE) inhibitors are among the most important drugs for treating patients with heart failure. ACE inhibitors open blood vessels and decrease the workload of the heart. Many studies suggest that ACE inhibitors may reduce the risk of death, heart attack, and hospital admissions by 28% in patients with existing heart failure.

Angiotensin-receptor blockers (ARBs): ARBs, also known as angiotensin II receptor antagonists, are similar to ACE inhibitors in their ability to open blood vessels and lower blood pressure.

Beta Adrenoceptor Antagonists (beta blockers): Beta blockers are almost always used in combination with other drugs such as ACE inhibitors and diuretics. They help slow heart rate and lower blood pressure.

Diuretics: Fluid retention is a major symptom of heart failure. Diuretics cause the kidneys to rid the body of excess salt and water. Aggressive use of diuretics can help eliminate excess body fluids, while reducing hospitalizations and improving exercise capacity. Diuretics are used in combination with other drugs, especially ACE inhibitors and beta blockers.

Aldosterone blockers: Aldosterone is a hormone that is critical in controlling the body's balance of salt and water. Excessive levels may play important roles in hypertension and heart failure.

Hydralazine and nitrates: Hydralazine and nitrates help relax arteries and veins, thereby reducing the heart's workload and allowing more blood to reach the tissues.

Statins: Statins are important drugs used to lower cholesterol and to prevent heart disease leading to heart failure, even in people with normal cholesterol levels.

Nesiritide: Nesiritide treats patients who have decompensated heart failure. Decompensated heart failure is a life-threatening condition in which the heart fails over the course of minutes or a few days, often as the result of a heart attack or sudden and severe heart valve problems.

Aspirin: Aspirin is a type of non-steroid anti-inflammatory (NSAID). A 2005 study in the Journal of the American College of Cardiology indicated that aspirin is important for preventing heart failure death in patients with heart disease, and can safely be used with ACE inhibitors. However, some studies have suggested that NSAIDs may increase the risk of heart failure for patients with a history of heart disease, especially when used in combination with ACE inhibitors or diuretics.

Additionally, heart surgery and interventional cardiology treatment with stents and catheters are used to unblock blood vessels to restore oxygen and nutrients to the heart. There are many device options for CHF therapy, such as devices that employ cardiac rhythm management (cardiac resynchronisation therapy—CRT) principles, which include cardiac resynchronization therapy pacemaker (CRT-P) and cardiac resynchronization therapy defibrillators (CRTD), ventricular assist devices (VAD), circulatory support devices, and mechanical support devices.

Most patients with HF are routinely managed with a combination of 3 types of drugs: a diuretic, an ACE Inhibitor or an ARB, and a beta-blocker. However, excessive use of diuretics can decrease blood pressure and impair renal function and exercise tolerance. The most common adverse effects of ACE inhibition in patients with HF are hypotension and dizziness. Sodium retention or depletion during long-term treatment with an ACEI can exaggerate or attenuate the cardiovascular and renal effects of treatment. Fluid retention can minimize the symptomatic benefits of ACE inhibition, whereas fluid loss increases the risk of hypotension and azotemia. Further, ACE inhibition may cause functional renal insufficiency.

In view of the foregoing there is a need to develop new therapeutics which will have the desired clinical effect without the above mentioned adverse effects. In particular, the ability to selectively regulate protein activity could provide an effective means to treat cardiovascular disease including congestive heart failure.

### SUMMARY OF THE INVENTION

The present invention relates to a method of identifying proteins implicated in cardiovascular disease, such as idiopathic cardiomyopathy, ischemic cardiomyopathy, dilated cardiomyopathy, cardiac hypertrophy and congestive heart failure. Preferably, the present invention provides for a method of identifying proteins implicated in congestive heart failure.

It is known that cardiovascular disease leading to heart failure involves not only contractile dysfunction, but also cardiomyocyte death. The present invention relates to the evaluation of microRNAs and their protein targets as potential therapeutic targets for the treatment of cardiovascular disease, in particular congestive heart failure. In particular, the present invention provides for candidate microRNAs and their protein targets that modulate ER stress-induced cardiomyocyte apoptosis.

In one aspect, the present invention provides for a method of identifying protein targets implicated in Endoplasmic Reticulum stress-induced cardiomyocyte apoptosis comprising:
(a) selecting at least one microRNA from the group consisting of miR-351, miR-322, miR-125, miR-424 and miR-7a; and
(b) identifying target genes of said microRNAs.

As used herein the term “protein targets implicated in Endoplasmic Reticulum stress-induced cardiomyocyte apoptosis” indicates proteins involved in the regulation of Endoplasmic Reticulum stress-induced cardiomyocyte apoptosis.

In the method of the present invention the microRNA may comprise miR-351 or miR-125. Alternatively, the microRNA may comprise miR-322 or miR-424. In a further embodiment, the microRNA may comprise miR-7a.

Within this specification the microRNAs miR-351, miR-322, miR-424 and miR-7a are oligonucleotides having the following sequences:

| | |
|---|---|
| (rno) miR-322 | cagcagcaauucauguuuugga |
| (hsa) miR-424 | cagcagcaauucauguuuugaa |
| (rno) miR-351 | ucccugaggagcccuuugagccuga |
| miR-7a | uggaagacuagugauuuuguugu |

miR-125 can represent either miR-125a or miR-125b the sequences of which are listed below:

| | |
|---|---|
| (hsa) miR-125a | ucccugagacccuuuaaccuguga |
| (hsa) miR-125b | ucccugagacccuaacuuguga |

According to the method of the present invention the step of identifying target genes of said microRNAs may comprise applying at least one computational algorithm to a gene database, wherein said computational algorithm selects genes which are implicated in apoptosis and cardiac function. Desirably, this comprises gene ontology analysis.

As used herein, the term “genes implicated in cardiac function” refers to those genes involved in regulating heart/cardiac processes.

The step of identifying target genes of said microRNAs according to the method of the present invention may further comprise applying at least one computational prediction algorithm to a gene database, wherein said computational prediction algorithm evaluates the ability of said microRNAs to bind specific mRNA targets of said genes. For the avoidance of any doubt, the term mRNA denotes messenger RNA. Suitably, the computational prediction algorithm comprises a bioinformatic algorithm.

In one embodiment of the method of the present invention the step of identifying target genes of said microRNAs comprises at least one step selected from the group consisting of:
(a) evaluating Watson-Crick base-pairing of said microRNA to a complementary mRNA site;
(b) evaluating the minimum free energy of the local microRNA-mRNA interaction;
(c) assessing the structural accessibility of the surrounding mRNA sequence; and
(d) combinations thereof,
wherein said mRNA is derived from said target gene.

The method of the present invention may further comprise the step of assessing evolutionary conservation of the 3′ untranslated region of mRNAs from said target genes and selecting those genes having evolutionary conserved target sites in the 3′ untranslated region of their corresponding mRNAs.

Preferably, the method of the present invention further comprises the step of selecting only those genes expressed in cardiomyocytes.

In one embodiment, the method of the present invention desirably comprises selecting those genes picked by two or more computational prediction algorithms.

In a further aspect the present invention provides for use of an oligonucleotide comprising sequence homology with at least one microRNA selected from the group consisting of miR-351, miR-322, miR-125, miR-424 and miR-7a for the treatment of cardiovascular disease. Desirably, said oligonucleotide will find use in the treatment of congestive heart failure. The oligonucleotide may comprise sequence homology with miR-351 or miR-125. Alternatively, the oligonucleotide may comprise sequence homology with miR-322 or miR-424. For example, the oligonucleotide may comprise sequence homology with miR-7a. Such oligonucleotides may also find use in the treatment of idiopathic cardiomyopathy, ischemic cardiomyopathy, dilated cardiomyopathy and cardiac hypertrophy.

In another aspect the present invention provides for use of an oligonucleotide comprising sequence homology with at least one microRNA selected from the group consisting of miR-351, miR-322, miR-125, miR-424 and miR-7a for regulating endoplasmic reticulum stress-induced apoptosis of cardiomyocytes. The oligonucleotide may comprise sequence homology with miR-351 or miR-125. Alternatively, the oligonucleotide may comprise sequence homology with miR-322 or miR-424. For example, the oligonucleotide may comprise sequence homology with miR-7a.

In yet a further aspect the present invention provides for a pharmaceutical composition for the treatment of cardiovascular disease comprising an oligonucleotide comprising sequence homology with at least one microRNA selected from the group consisting of miR-351, miR-322, miR-125, miR-424 and miR-7a together with a pharmaceutically acceptable carrier or excipients. Desirably, the pharmaceutical composition is for the treatment of congestive heart failure. The oligonucleotide may comprise sequence homology with miR-351 or miR-125. Alternatively, the oligonucleotide may comprise sequence homology with miR-322 or miR-424. For example, the oligonucleotide may comprise sequence homology with miR-7a. Such pharmaceutical compositions may also find use in the treatment of idiopathic cardiomyopathy, ischemic cardiomyopathy, dilated cardiomyopathy and cardiac hypertrophy.

As used herein the term “an oligonucleotide comprising sequence homology with” denotes an oligonucleotide with at least 75% sequence homology with one of miR-351, miR-322, miR-125, miR-424 and miR-7a. For example, greater than 80% sequence homology with one of miR-351, miR-322, miR-125, miR-424 and miR-7a. Such as, at least 85% sequence homology with one of miR-351, miR-322, miR-125, miR-424 and miR-7a. Desirably, greater than 90% sequence homology with one of miR-351, miR-322, miR-125, miR-424 and miR-7a. Further desirably, greater than 95% sequence homology with one of miR-351, miR-322, miR-125, miR-424 and miR-7a.

The invention also relates to a protein identified by the method of the present invention for the treatment of congestive heart failure.

The invention further relates to a protein identified by the method of the present invention for regulating endoplasmic reticulum stress-induced apoptosis of cardiomyocytes.

The invention further provides for a pharmaceutical composition for the treatment of cardiovascular disease comprising a protein identified by the method of the present invention together with a pharmaceutically acceptable carrier or excipients. Desirably, the pharmaceutical composition is for the treatment of congestive heart failure. Further uses may comprise the treatment of idiopathic cardiomyopathy, ischemic cardiomyopathy, dilated cardiomyopathy and cardiac hypertrophy.

The invention extends to a method of screening for candidate compounds for the treatment of cardiovascular disease (in particular congestive heart failure) or for regulating endoplasmic reticulum stress-induced apoptosis of cardiomyocytes comprising the steps of:
(a) identifying a protein target according to the method of the present invention;
(b) contacting said identified target protein with a test compound; and
(c) determining the effect of the test compound on said identified target protein.

Determining the effect of the test compound on the identified target protein may comprise determining if expression of the protein is up-regulated or down-regulated by the test compound. Alternatively, it may also comprise determining the effect of the test compound on the protein's function. Such as, inhibiting the regular function of the protein.

Where suitable, it will be appreciated that all optional and/or preferred features of one embodiment of the invention may be combined with optional and/or preferred features of another/other embodiment(s) of the invention.

### BRIEF DESCRIPTION OF THE DRAWINGS

Additional features and advantages of the present invention are described in, and will be apparent from, the detailed description of the invention and from the drawings in which:

FIG. 1 illustrates the RT-PCR results for induction of Grp78 with thapsigargin and tunicamycin in H9c2 cells; and

FIG. 2 illustrates a flow chart of the proposed microarray analysis.

### DETAILED DESCRIPTION OF THE INVENTION

The endoplasmic reticulum (ER) is a multifunctional signaling organelle that controls a wide range of cellular processes. The major physiological functions of ER include folding of membrane and secreted proteins, synthesis of lipids and sterols, and storage of free calcium. Cellular stresses that impair proper folding of proteins can lead to an imbalance between the load of resident and transit proteins in the ER and the organelle's ability to process that load. In mammals, three ER transmembrane proteins, IRE1, ATF6, and PERK, respond to the accumulation of unfolded proteins in the ER lumen. Activation of PERK, IRE1, and ATF6 initiates ER-to-nucleus intracellular signalling cascades collectively termed as unfolded protein response (UPR). The most salient feature of UPR is to increase the transactivation function of a plurality of bZIP transcription factors, such as ATF6, ATF4 and XBP1. Once activated, these transcription factors coordinate transcriptional induction of ER chaperones and genes involved in ER-associated degradation (ERAD) to enhance the protein folding capacity of the cell and to decrease the unfolded protein load of the ER, respectively.

However, if the damage is irreparable and ER homeostasis cannot be restored, the mammalian UPR ultimately initiates apoptosis. The exact mechanism involved in transition of the UPR from protective to apoptotic is not clearly understood. A class of small RNAs, known as microRNAs, have been shown to be critically involved in control of cell survival and cell death decisions. MicroRNAs are generated from RNA transcripts that are exported into the cytoplasm, where the primary-microRNA molecules undergo Dicer-mediated processing to generate mature microRNA. The mature microRNAs assemble into ribonucleoprotein silencing complexes (RISCs) and guide the silencing complex to specific mRNA molecules. MicroRNAs direct posttranscriptional regulation of gene expression, typically by binding to 3′ UTR of cognate mRNAs and inhibiting their translation and/or stability.

Hundreds of microRNAs, many of them evolutionarily conserved, have been identified in mammals, but their physiological functions are just beginning to be elucidated. Several studies have shown global alterations in microRNA-expression profiles during various types of cellular stresses, such as folate deficiency, arsenic exposure, hypoxia, drug treatment and genotoxic stress.

In particular, the present inventors have evaluated microRNAs and their protein targets as potential therapeutic targets for the treatment of congestive heart failure.

### Approach

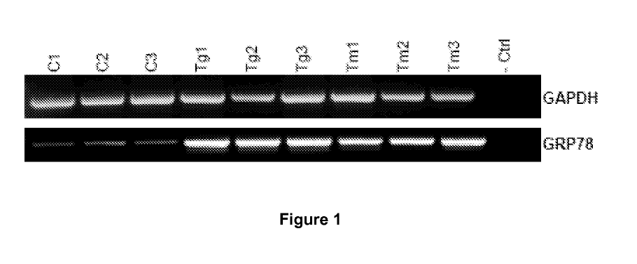

Expression profiling of microRNAs during the conditions of Endoplasmic Reticulum (ER) stress in cardiomyocytes was performed. ER stress was induced by treatment with either thapsigargin, an inhibitor of the Sacroplasmic/Endoplasmic Reticulum Ca2+ATPase (SERCA) pump or tunicamycin (an inhibitor of N-linked glycosylation). RNA was isolated from three independent experiments where H9c2 cells were treated with thapsigargin (Tg) or tunicamycin (Tm) for 24 hours. RNAs from Tg and Tm treated cells were checked for induction of key ER resident chaperone Grp78/BiP by RT-PCR. Grp78/BiP is a central regulator of ER homeostasis due to its multiple functional roles in protein folding, ER calcium binding, and controlling of the activation of transmembrane ER stress sensors. As shown in FIG. 1, RT-PCR analysis of Tg and Tm treatment led to induction of Grp78/BiP in all three experiments. Total RNA was isolated from H9c2 cells treated with 1 μM Tg, 1 μg/ml Tm for 24 hr and the expression levels of the indicated genes were analysed by RT-PCR. The control experiments labelled C1-C3 do not show induction of Grp78/BiP.

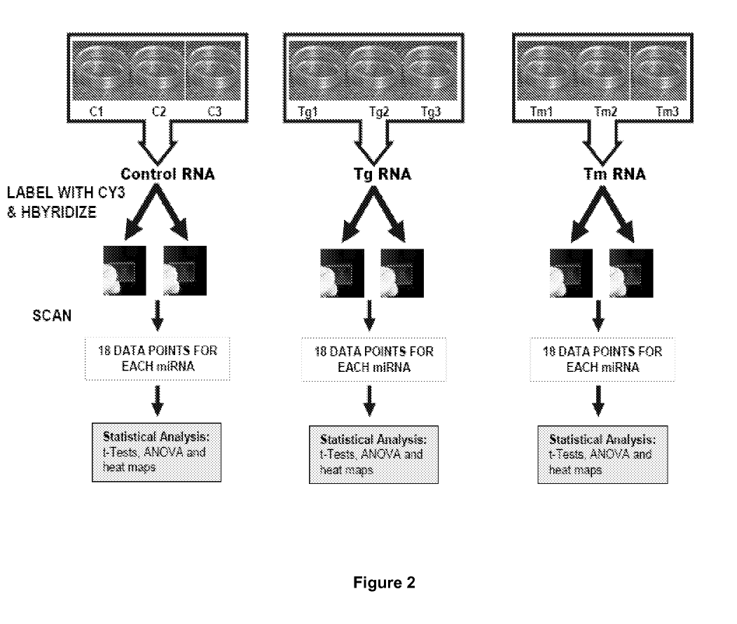

Next equal amounts of RNAs from each experiment were pooled and used for microarray analysis to minimize experimental variations. The experimental outline for the microarray analysis is illustrated in FIG. 2. The chips were spotted with 350 mature microRNAs of Rat as per Sanger miRBase database (Release 11.0). Each microRNA was spotted on the array nine times and for each RNA sample two chips were used. There were 16 sets of control probes on each array. There were greater than 10 positive controls (spike-in controls & 5S). There were greater than 10 negative controls (mismatch control). A 20-mer control RNA is spiked into each sample followed by labeling and hybridization. The control RNA had been computationally and experimentally verified not to cross-hybridize with the probes of any known microRNA transcript. The background-subtracted signals were used for statistical tests and clustering analysis.

### Results

Microarray analysis showed that out of 350 microRNAs spotted per chip, on average 198 microRNAs were detected. Further we found that expression of 109 microRNAs changed significantly during conditions of ER stress in H9c2 cardiomyocytes. We observed significant upregulation of mir-125, mir-126, let-7b and let-7c whereas substantial downregulation of mir-20a, mir-17, mir-93, mir-206, mir-133a and mir-133b. A similar alteration in expression level of these microRNAs has been previously reported during conditions of idiopathic cardiomyopathy, ischemic cardiomyopathy, dilated cardiomyopathy, cardiac hypertrophy and heart failure. The ample overlap of microRNA expression signature between our analysis (in ER stress conditions) and different models of cardiac dysfunction further confirms the role of ER stress in cardiomyocyte apoptosis.

TABLE 1

| | Microarray | | | Real-time | | | Real-time | | |
|---|---|---|---|---|---|---|---|---|---|
| | Analysis | | | PCR (set I) | | | PCR (set II) | | |
| | con- | | | con- | | | con- | | |
| | trol | Tg | Tm | trol | Tg | Tm | trol | Tg | Tm |
| mir-98 | 1.000 | 3.822 | 4.921 | 1.000 | 1.470 | 1.480 | 1.000 | 0.850 | 0.750 |
| mir-7a | 1.000 | 1.341 | 2.458 | 1.000 | 1.540 | 1.140 | 1.000 | 3.800 | 1.650 |
| mir-24 | 1.000 | 0.664 | 0.672 | 1.000 | 0.810 | 0.930 | 1.000 | 0.870 | 0.760 |
| mir-25 | 1.000 | 0.633 | 0.771 | 1.000 | 0.790 | 0.906 | 1.000 | 0.799 | 0.760 |
| mir-351 | 1.000 | 0.141 | 0.179 | 1.000 | 0.210 | 0.290 | 1.000 | 0.210 | 0.230 |
| mir-322 | 1.000 | 0.094 | 0.113 | 1.000 | 0.830 | 0.840 | 1.000 | 0.690 | 0.610 |
| mir-20a | 1.000 | 0.770 | 0.791 | 1.000 | 0.930 | 1.340 | 1.000 | 0.620 | 0.650 |
| mir-107 | 1.000 | 0.682 | 0.601 | 1.000 | 0.580 | 0.590 | 1.000 | 0.800 | 0.630 |
| mir-103 | 1.000 | 0.654 | 0.571 | 1.000 | 0.860 | 0.960 | 1.000 | 0.590 | 0.500 |
| mir-93 | 1.000 | 0.583 | 0.524 | 1.000 | 0.660 | 0.800 | 1.000 | 0.530 | 0.550 |
| mir- | 1.000 | 0.638 | 0.672 | 1.000 | 0.820 | 1.060 | 1.000 | 0.800 | 0.737 |
| 106b | | | | | | | | | |
| mir-206 | 1.000 | 0.683 | 0.607 | 1.000 | 0.540 | 0.570 | 1.000 | 0.820 | 0.604 |
| mir-18a | 1.000 | 0.426 | 0.550 | 1.000 | 0.720 | 1.090 | 1.000 | 0.700 | 0.640 |
| mir- | 1.000 | 0.565 | 0.641 | 1.000 | 0.470 | 0.480 | 1.000 | 0.680 | 0.510 |
| 133b | | | | | | | | | |
| mir- | 1.000 | 0.595 | 0.690 | 1.000 | 0.720 | 0.750 | 1.000 | 1.140 | 0.730 |
| 133a | | | | | | | | | |

### Confirmation of Results by Reverse Transcription PCR:

Further differential expression of 16 microRNAs has been confirmed by real-time RT-PCR (2 upregulated and 14 down regulated). Expression of muscle specific microRNAs; mir-206, mir-133a and mir-133b and several members of mir-17-92 oncogenic cluster were repressed during conditions of ER stress. Based on their differential expression profile during ER stress and their hitherto unexplored role in cardiovascular biology mir-7a, mir-351 and mir-322 were identified as primary microRNA targets in conditions of ER stress. In addition, the invention has been extended to the human ortholog, miR-125, of rat miR-351. Similarly, the invention extends to the human ortholog, miR-424, of rat miR-322. hsa-miR-351 & rno-miR-125, and hsa-miR-424 & rno-miR-322 are microRNAs having similar seed sequences in humans and rats respectively. Logically, these microRNA pairs would possess functional equivalence in regulating the expression of similar genes in humans and rats respectively.

Table 1 shows the List of microRNAs showing altered expression during conditions of ER stress in H9c2 cardiomyocytes. Control, Untreated; Tg, thapsigargin (1 μM) for 24 hours, Tm, tunicamycin (1 μg/ml) for 24 hours. mir-7a, mir-351 and mir-322 are shown in bold face.

### Bioinformatics Analysis:

Most of the genome wide analysis generates a list of few hundreds of genes. The thorough experimental testing of such vast numbers of predicted targets using labour intensive transgenic reporter assays is impractical. A combination of computational and Gene Ontology (GO) analysis to compile a list of functionally relevant target genes of mir-7a, mir-351 and mir-322 has been employed.

Many computational methods have been developed to predict microRNA targets. The criteria for target prediction vary widely, but often include:
(i) strong Watson-Crick base-pairing of the 5′ seed of the microRNA (nucleotide positions 2-8 of the microRNA) to a complementary site in the 3′ untranslated region (UTR) of the mRNA;
(ii) conservation of the microRNA binding site;
(iii) favourable minimum free energy (MFE) for the local microRNA-mRNA interaction; and
(iv) structural accessibility of the surrounding mRNA sequence.

Three bioinformatic algorithms, miRANDA, TargetScan and PicTar were employed to predict respective microRNA target genes. The genes which were picked up by more than one algorithm and having evolutionary conserved target sites in their 3′UTRs were selected. However the microRNA and its target mRNA must be co-expressed in order for the microRNA to repress the expression of its biological target. Therefore the list was amended to exclude the genes whose expression has not been reported in cardiomyocytes. The list was further edited to include only those genes which overlapped with GO terms such as Heart processes and apoptosis. As shown in table II, III and IV, in addition to genes know to affect apoptosis pathways, the tables contain several protein phosphatases, potassium and sodium ion channels and gap junction proteins. Altered expression of these proteins is likely to play a crucial role during cardiovascular dysfunctions.

TABLE 2

| Human | |
|---|---|
| ortholog | Gene name |
| RB1 | retinoblastoma 1 (including osteosarcoma) |
| RAF1 | v-raf-1 murine leukemia viral oncogene homolog 1 |
| BCLW | Bcl2-like2 |
| ITCH | itchy homolog E3 ubiquitin protein ligase (mouse) |
| BIRC4 | baculoviral IAP repeat-containing 4 |
| TMSB4X | thymosin, beta 4 |
| ERBB4 | v-erb-a erythroblastic leukemia viral oncogene homolog 4 |
| | (avian) |
| DDIT4 | DNA-damage-inducible transcript 4 |
| VDAC1 | voltage-dependent anion channel 1 |
| VDAC3 | voltage-dependent anion channel 3 |
| IGF2BP2 | insulin-like growth factor 2 mRNA binding protein 2 |
| IRS2 | insulin receptor substrate 2 |
| PLCB1 | phospholipase C, beta 1 (phosphoinositide-specific) |
| PTP4A1 | protein tyrosine phosphatase 4a1 |
| Dusp2 | dual specificity phosphatase 2 |
| PPP2R2D | protein phosphatase 2, regulatory subunit B, delta isoform |
| PTPNS1 | protein tyrosine phosphatase, non-receptor type substrate 1 |
| PTPRD | protein tyrosine phosphatase, receptor type, D |
| Dusp9 | dual specificity phosphatase 9 |
| PPM1B | protein phosphatase 1B, magnesium dependent, beta isoform |
| PPP2R1B | protein phosphatase 2 (formerly 2A), regulatory subunit A |
| | (PR 65), beta isoform |
| PPP1CA | protein phosphatase 1, catalytic subunit, alpha isoform |
| KCNH5 | potassium voltage-gated channel, subfamily H (eag-related), |
| | member 5 |
| KCNJ2 | potassium inwardly-rectifying channel, subfamily J, member 2 |
| KCNJ2 | potassium inwardly-rectifying channel, subfamily J, member 2 |
| KCNC3 | potassium voltage gated channel, Shaw-related subfamily, |
| | member 3 |
| SCN2B | sodium channel, voltage-gated, type II, beta |
| ATP2B2 | ATPase, Ca++ transporting, plasma membrane 2 |
| TCF12 | transcription factor 12 (HTF4, helix-loop-helix transcription |
| | factors 4) |
| THRAP2 | thyroid hormone receptor associated protein 2 |
| GJA5 | gap junction membrane channel protein alpha 5 |
| GLI3 | GLI-Kruppel family member GLI3 |
| | (Gneig cephalopolysyndactyly syndrome) |
| SFRS1 | splicing factor, arginine/serine-rich 1 (splicing factor 2, |
| | alternate splicing factor) |
| SRF | serum response factor (c-fos serum response element- |
| | binding transcrption factor) |

Table 2 lists the human ortholog of rno-mir-7a target genes having evolutionary conserved target sites in their 3′ UTRs, which are expressed in heart and are predicted to affect important heart functions.

Table 3 lists the human ortholog of rno-mir-351 target genes having evolutionary conserved target sites in their 3′ UTRs, which are expressed in heart and are predicted to affect important heart functions.

Table 4 lists the human ortholog of rno-mir-322 target genes having evolutionary conserved target sites in their 3′ UTRs, which are expressed in heart and are predicted to affect important heart functions.

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

TABLE 3

| Human | |
|---|---|
| ortholog | Gene name |
| TAZ | tafazzin (cardiamyopathy dilated 3A (X-linked); endocardial fibroelastosis 2; Barth syndrome) |
| LBH | limb bud and heart development homolog (mouse) |
| BMF | Bcl2 modifying factor |
| BAK1 | BCL2-antagonist/killer 1 |
| BCLW | BCL2-LIKE 2 |
| PTPN18 | protein tyrosine phosphatase, non-receptor type 18 (brain-denved) |
| PPP2R1B | protein phosphatase 2 (formerly 2A), regulatory subunit A (PR 65), beta isoform |
| PPP2CA | protein phosphatase 2 (formerly 2A), catalytic subunit, alpha isoform |
| PPP1CA | protein phosphatase 1, catalytic subunit, alpha isoform |
| PPP2R5C | protein phosphatase 2, regulatory subunit B′, gamma isoform |
| PPP2B3A | protein phosphatase 2 (formerly 2A), regulatory subunit B″, alpha |
| DUSP6 | dual specificity phosphatase 6 |
| ATP1B4 | ATPase, (Na+)/K+ transporting, beta 4 polypeptide |
| HCN4 | hyperpolarization activated cyclic nucleotide-gated potassium channel 4 |
| SCN4B | sodium channel, voltage-gated, type IV, beta |
| SCN5A | sodium channel, voltage-gated, type V, alpha subunit |
| KCNS3 | potassium voltage-gated channel, delayed-rectifier, subfamily S, member 3 |
| KCNA1 | potassium voltage-gated channel, shaker-related subfamily, member 1 (episodic ataxia with |
| | myokymia) |
| KCNJ12 | potassium inwardly-rectifying channel, subfamily J, member 12 |
| KCNJ11 | potassium inwarldy-rectifying channel, subfamily J, member 11 |
| KCNIP2 | Kv channel-interacting protein 2 |
| KCNIP3 | Kv channel interacting protein 3, calsenilin |
| KCTD21 | potassium channel tetramerisation domain containing 21 |
| GJA1 | gap junction protein, alpha 1, 43 kDa |
| GJA5 | gap junction membrane channel protein alpha 5 |
| ACVR2A | activin A receptor, type IIA |
| SLC8A2 | solute carrier family 8 (sodium-calcium exchanger), member 2 |
| ERBB4 | v-erb-a erythroblastic leukemia viral oncogene homolog 4 (avian) |
| FGFR2 | fibroblast growth factor receptor 2 (bacteria-expressed kinase, keralinocyte growth factor receptor, |
| | craniofacial dysosiosis 1, Crouzon syndrome, Pleiffer syndrome, Jackson-Weiss syndrome) |
| NUMBL | numb homolog (Drosophila)-like |
| EDN1 | endothelin 1 |
| TGFBR1 | transforming growth factor, beta receptor I (activin A receptor type II-like kinase, 53 kDa) |
| DVL3 | dishevelied, dsh homolog 3 (Drosophila) |
| SRF | serum response factor (c-fos serum response element-binding transcription factor) |
| MEF2D | MADS box transcription enhancer factor 2, polypeptide D (myocyte enhancer factor 2D) |

TABLE 4

| Human | |
|---|---|
| ortholog | Gene name |
| BCL2 | B-cell CLL/lymphoma 2 |
| BCLW | BCL2-like 2 |
| BFAR | bifunctional apoptosis regulator |
| PDCD4 | programmed cell death 4 (neoplastic transformation |
| | inhibitor) |
| DFDD | death effector domain containing |
| CARD10 | caspase recruitment domain family, member 10 |
| BCL9L | B-cell CLL/lymphoma 9-like |
| PPP1R12B | protein phosphatase 1, regulatory (inhibitor) subunit 12B |
| PPP3CB | protein phosphatase 3 (formerly 2B), catalytic subunit, |
| | beta isoform |
| PPP2R1A | protein phosphatase 2 (formerly 2A), regulatory subunit |
| | A, alpha isoform |
| PPP6C | protein phosphatase 6, catalytic subunit |
| PPP2R5C | protein phosphatase 2, regulatory subunit B′, gamma |
| | isoform |
| DUSP3 | dual specificity phosphatase 3 (vaccinia virus phosphatase |
| | VH1-related) |
| PPF1A3 | protein tyrosine phosphatase, receptor type, f polypeptide |
| | (PTPRF), interacting protein (liprin), alpha 3 |
| CALM1 | calmodulin 1 (phosphorylase kinase, delta) |
| PIM1 | pim-1 oncogene |
| MAP2K3 | mitogen-activated protein kinase kinase 3 |
| PRKACA | protein kinase, cAMP-dependent, catalytic, alpha |
| KCNJ2 | potassium inwardly-rectifying channel, subfamily J, |
| | mermber 2 |
| KCNAB1 | potassium voltage-gated channel, shaker-related subfamily, |
| | beta member 1 |
| KCTD8 | potassium channel tetramerisation domain containing 8 |
| KCTD1 | potassium channel tetramerisation domain containing 1 |
| KCND5 | potassium voltage-gated channel, KQT-like subfamily, |
| | member 5 |
| SCN4B | sodium channel, voltage-gated, type IV, beta |
| CACNB1 | calcium channel, voltage-dependent, beta 1 subunit |
| ATP1B4 | ATPase, (Na+)/K+ transporting, beta 4 polypeptide |
| ABCC5 | ATP-binding cassette, sub-family C (CFTR/MRP), |
| | member 5 |
| IGF1R | insulin-like growth factor 1 receptor |
| IGF2R | insulin-like growth factor 2 receptor |
| IPPK | inositol 1,3,4,5,6-pentakisphosphate 2-kinase |
| ITPR1 | inositol 1,4,5-triphosphate receptor, type 1 |
| THRAP1 | thyroid hormone receptor associated protein 1 |
| SEMA3D | sema domain, immunoglobulin domain (Ig), short basic |
| | domain, secreted, (semaphorin) 3D |
| SEMA3A | sema domain, immunoglobulin domain (Ig), short basic |
| | domain, secreted, (semaphorin) 3A |
| NRP2 | neuropilin 2 |
| SMAD5 | SMAD family member 5 |
| ACVR2B | activin A receptor, type IIB |
| ACVR2A | activin A receptor, type IIA |
| RARB | retinoic acid receptor, beta |
| FZD10 | frizzled homolog 10 (Drosophila) |
| GNAI3 | guanine nucleotide binding protein (G protein), alpha |
| | inhibiting activity polypeptide 3 |
| ADRB2 | adrenergic, beta-2-, receptor, surface |
| CRKL | v-crk sarcoma virus CT10 onocgene homolog (avian)-like |
| BMPR1A | bone morphogenetic protein receptor, type IA |
| PDLIM5 | PDZ and LIM domain 5 |
| APLN | apelin, AGTRL1 ligand |
| NFATC3 | nuclear factor of activated T-cells, cytoplasmic, calcineurin- |
| | dependent 3 |
| SGCD | sarcoglycan, delta (35 kDa dystrophin-associated |
| | glycoprotein) |

1. A method of identifying protein targets implicated in Endoplasmic Reticulum stress induced cardiomyocyte apoptosis comprising:
(a) selecting at least one microRNA from the group consisting of miR-351, miR-322, miR-125, miR-424 and miR-7a; and
(b) identifying target genes of said microRNAs.

2. A method according to claim 1 wherein the microRNA comprises miR-351 or miR-125.

3. A method according to claim 1 wherein the microRNA comprises miR-322 or miR-424.

4. A method according to claim 1 wherein the microRNA comprises miR-7a.

5. A method according to claim 1 wherein the step of identifying target genes of said microRNAs comprises applying at least one computational algorithm to a gene database, wherein
said computational algorithm selects genes which are implicated in apoptosis and cardiac function.

6. A method according to claim 1 wherein the step of identifying target genes of said microRNAs comprises applying at least one computational prediction algorithm to a gene database, wherein
said computational prediction algorithm evaluates the ability of said microRNAs to bind specific mRNA targets of said genes.

7. A method according to claim 1 wherein the step of identifying target genes of said microRNAs comprises a step selected from the group consisting of:
(a) evaluating Watson-Crick base-pairing of said microRNA to a complementary mRNA site;
(b) evaluating the minimum free energy of the local microRNA-mRNA interaction;
(c) assessing the structural accessibility of the surrounding mRNA sequence; and
(d) combinations thereof,
wherein said mRNA is derived from said target gene.

8. A method according to claim 5 further comprising the step of assessing evolutionary conservation of the 3′ untranslated region of mRNAs from said target genes and selecting those genes having evolutionary conserved target sites in the 3′ untranslated region of their corresponding mRNAs.

9. A method according to claim 5 further comprising the step of selecting only those genes expressed in cardiomyocytes.

10. A method according to claim 6 comprising selecting those genes picked by two or more computational prediction algorithms.

11. An oligonucleotide comprising sequence homology with at least one microRNA selected from the group consisting of miR-351, miR-322, miR-125, miR-424 and miR-7a for use in the treatment of cardiovascular disease.

12. An oligonucleotide according to claim 11 for use in the treatment of congestive heart failure.

13. An oligonucleotide comprising sequence homology with at least one microRNA selected from the group consisting of miR-351, miR-322, miR-125, miR-424 and miR-7a for use in regulating endoplasmic reticulum stress-induced apoptosis of cardiomyocytes.

14. A pharmaceutical composition for the treatment of cardiovascular disease comprising an oligonucleotide comprising sequence homology with at least one microRNA selected from the group consisting of miR-351, miR-322, miR-125, miR-424 and miR-7a together with a pharmaceutically acceptable carrier or excipients.

15. A pharmaceutical composition according to claim 14 for the treatment of congestive heart failure.

16. A protein identified by the method of claim 1 for use in the treatment of cardiovascular disease.

17. A protein according to claim 16 for use in the treatment of congestive heart failure.

18. A protein identified by the method of claim 1 for use in regulating endoplasmic reticulum stress-induced apoptosis of cardiomyocytes.

19. A pharmaceutical composition for the treatment of cardiovascular disease comprising a protein according to claim 16 together with a pharmaceutically acceptable carrier or excipients.

20. A pharmaceutical composition according to claim 19 for the treatment of congestive heart failure.

21. A method of screening for candidate compounds for the treatment of congestive heart failure or for regulating endoplasmic reticulum stress-induced apoptosis of cardiomyocytes comprising the steps of:
(a) Identifying a protein target according to the method of claim 1;
(b) contacting said identified target protein with a test compound; and
(c) determining the effect of said test compound on said identified target protein.
